(19) United States
(12) Patent Application Publication
(10) Pub. No.: US 2016/0051130 A1
(43) Pub. Date: Feb. 25, 2016

(54) DISTAL END CAP FOR ENDOSCOPE AND ENDOSCOPE APPARATUS

(71) Applicant: OLYMPUS CORPORATION, Tokyo (JP)

(72) Inventor: Tae MITSUYA, Machida-shi (JP)

(21) Appl. No.: 14/928,763

(22) Filed: Oct. 30, 2015

Related U.S. Application Data
(63) Continuation of application No. PCT/JP2014/078789, filed on Oct. 29, 2014.

(30) Foreign Application Priority Data
Nov. 15, 2013 (JP) 2013-237432

(51) Int. Cl. A61B 1/00; A61B 1/06

(52) U.S. Cl. CPC A61B 1/00137; A61B 1/00096; A61B 1/0676; A61B 1/00101

(57) ABSTRACT

A distal end cap for an endoscope includes a cap proximal end portion, a cap distal end portion, a concave portion, a low-strength portion and at least one high-strength portion. The low-strength portion is disposed on a same line as the concave portion in an axial direction and continuously disposed between the cap distal end portion and the cap proximal end portion in a axial direction. The high-strength portion has strength higher than that of the low-strength portion, disposed on at least a portion excluding the concave portion in an axis circumferential direction of the axis, and continuously disposed from the cap distal end portion to the cap proximal end portion in the axial direction.

### CROSS-REFERENCE TO RELATED APPLICATIONS

This application is a Continuation application of PCT Application No. PCT/JP2014/078789, filed Oct. 29, 2014 and based upon and claiming the benefit of priority from prior Japanese Patent Application No. 2013-237432, filed Nov. 15, 2013, the entire contents of all of which are incorporated herein by reference.

### BACKGROUND OF THE INVENTION

1. Field of the Invention

The present invention relates to a distal end cap for an endoscope and an endoscope apparatus.

2. Description of the Related Art

When an insertion portion of an endoscope is inserted into the stomach, for example, gastric mucosa may adhere to an observation window portion disposed on a distal end portion of the insertion portion. Therefore, a desired visual field may not be ensured. For this reason, a distal end cap (hereinafter referred to as a cap) for an endoscope to prevent the adhesion of mucosa to the observation window portion is attached to the distal end portion of the insertion portion.

Such caps are disclosed in, for example, Jpn. Pat. Appln. KOKAI Publication No. 11-313795, Jpn. Pat. Appln. KOKAI Publication No. 2003-116772, Jpn. Pat. Appln. KOKAI Publication No. 2006-325816, and Jpn. Pat. Appln. KOKAI Publication No. 2001-224550.

### BRIEF SUMMARY OF THE INVENTION

An aspect of a distal end cap for an endoscope of the present which is removably attached in an axial direction to an insertion end portion of an insertion portion disposed in the endoscope, the distal end cap including: a cap proximal end portion which is disposed at a proximal end side of the distal end cap in the axial direction and attached to the insertion end portion; a cap distal end portion which is disposed at a distal end side of the distal end cap in the axial direction, and is disposed at a position so that the cap distal end portion protrudes from an distal end surface of the insertion end portion when the cap proximal end portion is attached to the insertion end portion; a concave portion which is disposed on the cap distal end portion from the distal end side toward the proximal end side in the axial direction; a low-strength portion which is disposed on a same line as the concave portion in the axial direction and continuously disposed between the cap distal end portion and the cap proximal end portion in the axial direction; and at least one high-strength portion having strength higher than that of the low-strength portion, the high-strength portion being disposed on at least a portion excluding the concave portion in an axis circumferential direction of an axis, and continuously disposed from the cap distal end portion to the cap proximal end portion in the axial direction.

Advantages of the invention will be set forth in the description which follows, and in part will be obvious from the description, or may be learned by practice of the invention. Advantages of the invention may be realized and obtained by means of the instrumentalities and combinations particularly pointed out hereinafter.

### BRIEF DESCRIPTION OF DRAWINGS

The accompanying drawings, which are incorporated in and constitute apart of the specification, illustrate embodiments of the invention, and together with the general description given above and the detailed description of the embodiments given below, serve to explain the principles of the invention.

FIG. 1A is a schematic perspective view of an endoscope apparatus according to a first embodiment of the present invention.

FIG. 1B is a perspective view of a distal end cap for an endoscope attached to a distal end hard portion.

FIG. 1C is a perspective view of the distal end cap for an endoscope illustrated in FIG. 1B.

FIG. 1D is a diagram illustrating an observation visual field obtained when the distal end cap for an endoscope illustrated in FIG. 1C is attached to the distal end hard portion.

FIG. 1E is a perspective view of another cylindrical distal end cap for an endoscope different from the distal end cap for an endoscope of the present embodiment.

FIG. 1F is a diagram illustrating an observation visual field obtained when the distal end cap for an endoscope illustrated in FIG. 1E is attached to the distal end hard portion.

FIG. 2A illustrates an example of a high-strength portion and is a front view of the distal end cap for an endoscope attached to the distal end hard portion.

FIG. 2B illustrates an example of a high-strength portion and is a front view of the distal end cap for an endoscope attached to the distal end hard portion.

FIG. 2C illustrates an example of a high-strength portion and is a front view of the distal end cap for an endoscope attached to the distal end hard portion.

FIG. 3A is a perspective view of slip prevention portions and a marker.

FIG. 3B is a perspective view of slip prevention portions and a marker.

FIG. 3C is a perspective view of a penetration prevention portion.

FIG. 4 is a side view of a distal end cap for an endoscope of a first modification.

FIG. 5 is a side view of a distal end cap for an endoscope of a second modification.

FIG. 6 is a front view of a distal end cap for an endoscope of a third modification.

FIG. 7 is a front view of a distal end cap for an endoscope of a fourth modification.

FIG. 8A is a front view of a distal end cap for an endoscope of a fifth modification.

FIG. 8B is a front view of a distal end cap for an endoscope of the fifth modification.

FIG. 9A is a perspective view of a distal end cap for an endoscope of a sixth modification.

FIG. 9B is a perspective view of a distal end cap for an endoscope of the sixth modification.

### DETAILED DESCRIPTION OF THE INVENTION

The following describes embodiments of the present invention with reference to the accompanying drawings. Note that some drawings omit illustrating some parts in order to clarify the illustration.

### First Embodiment

### Configuration

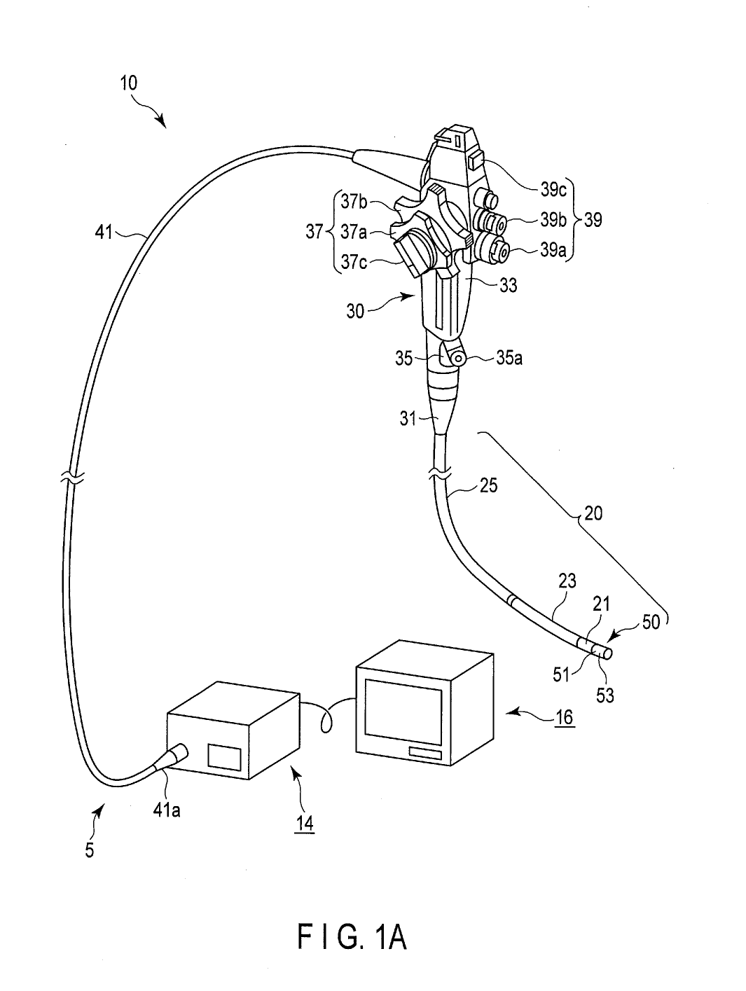

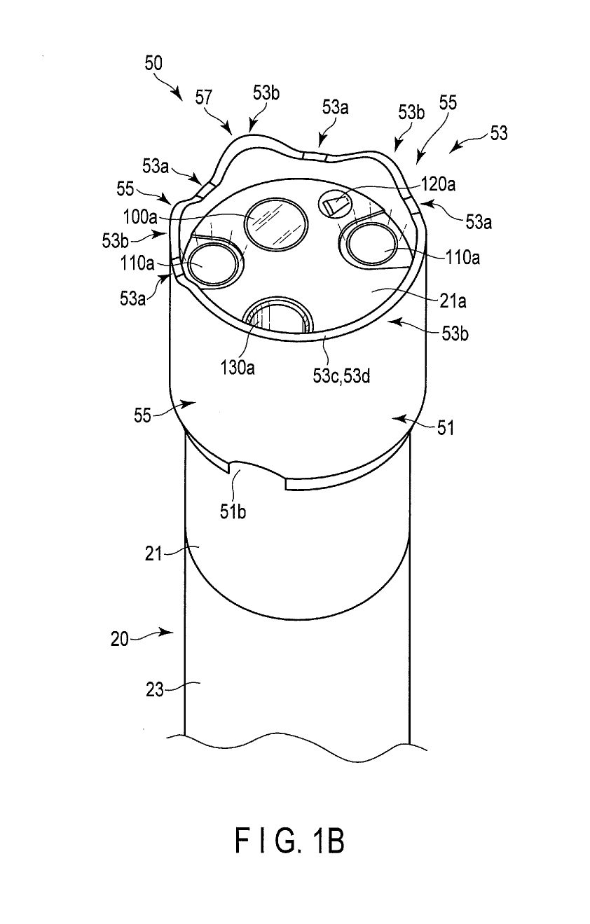

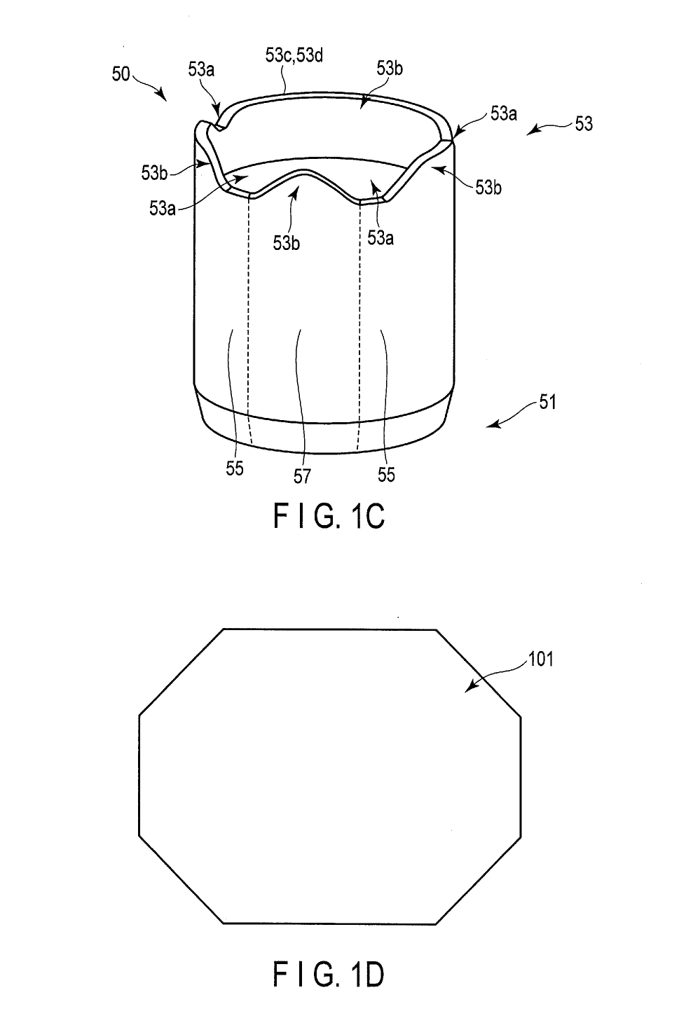

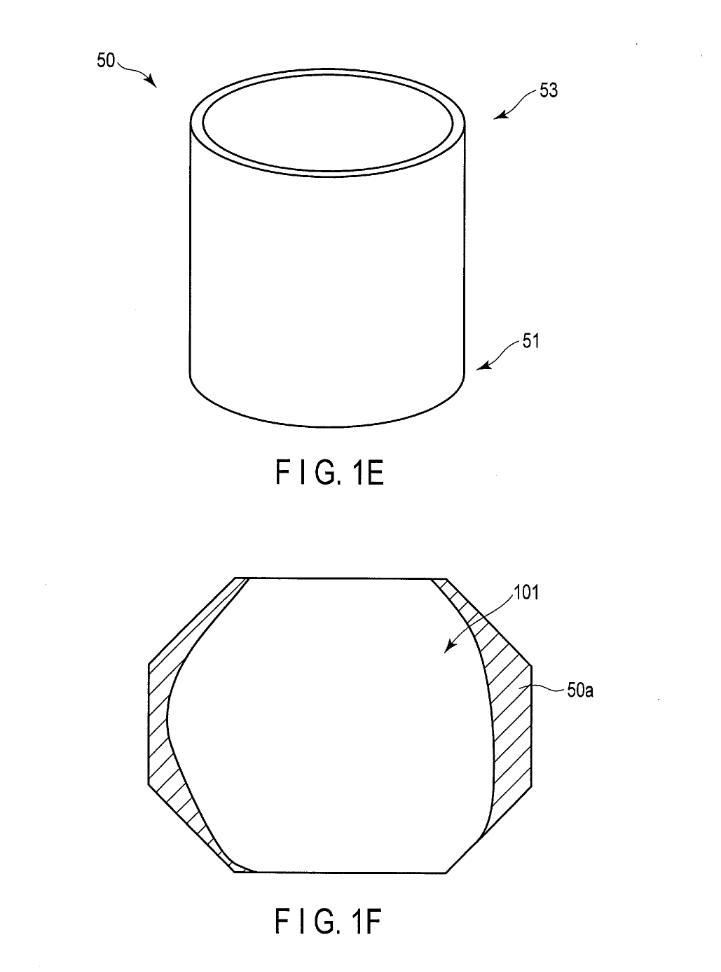

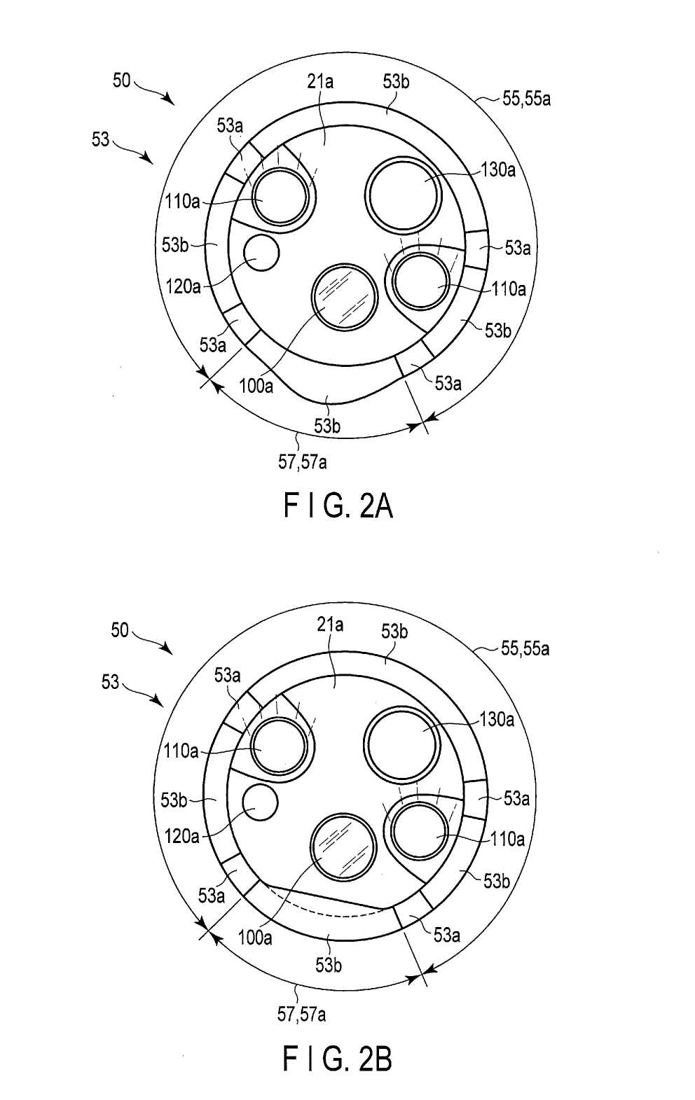

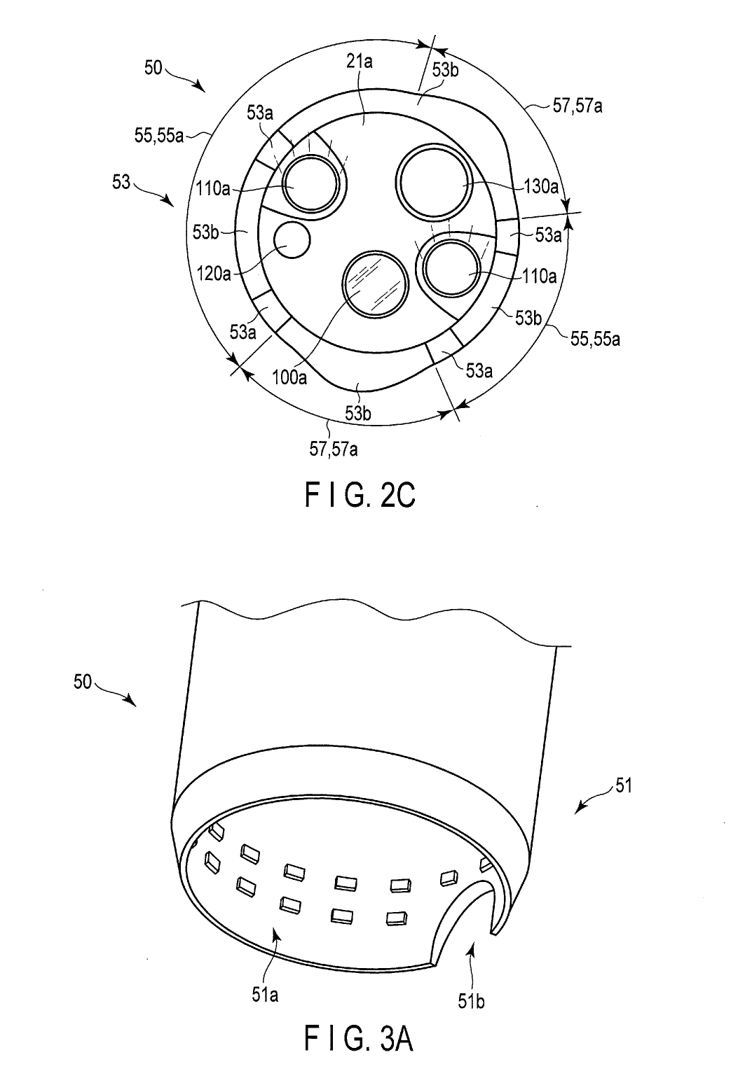

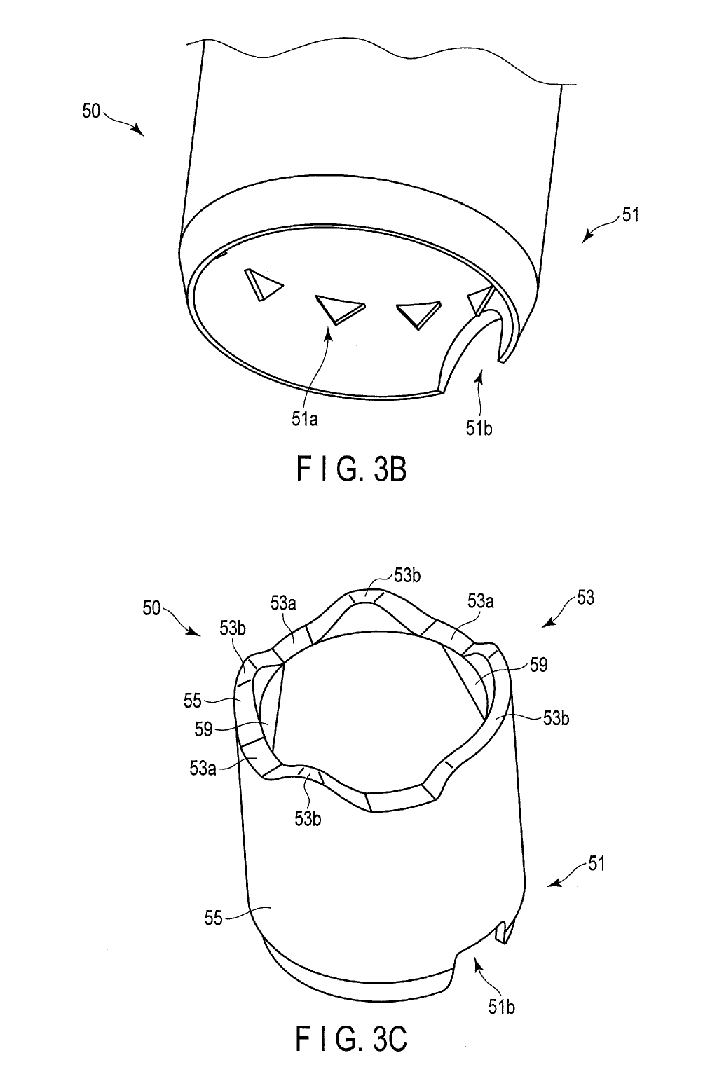

A first embodiment is described with reference to FIG. 1A, FIG. 1B, FIG. 1C, FIG. 1D, FIG. 1E, FIG. 1F, FIG. 2A, FIG. 2B, FIG. 2C, FIG. 3A, FIG. 3B, and FIG. 3C.

[Endoscope Apparatus 5]

As illustrated in FIG. 1, an endoscope apparatus 5 includes an endoscope 10 including an image capture unit (not illustrated), a control apparatus 14 which control the endoscope 10, and a display portion 16 connected to the control apparatus 14. The control apparatus 14 includes an image process portion (not illustrated) which process images photographed by the image capture unit. The display portion 16 is configured to display the images photographed by the image capture unit and processed by the image processor portion.

[Endoscope 10]

An endoscope 10 illustrated in FIG. 1A is an example of a direct viewing endoscope.

As illustrated in FIG. 1A, for example, the endoscope 10 includes an insertion portion 20 which has a long and narrow shape and is to be inserted in, for example, the lumen of a patient and an operation portion 30 which is coupled to a proximal end portion of the insertion portion 20 and operates the endoscope 10.

[Insertion Portion 20]

As illustrated in FIG. 1A, the insertion portion 20 includes in this order a distal end hard portion 21, a bending portion 23, and a flexible tube portion 25 from a distal end portion side of the insertion portion 20 to the proximal end portion side of the insertion portion 20. A proximal end portion of the distal end hard portion 21 is coupled to a distal end portion of the bending portion 23. A proximal end portion of the bending portion 23 is coupled to a distal end portion of the flexible tube portion 25.

The distal end hard portion 21 is the distal end portion of the insertion portion 20 and is hard and inflexible. The configuration of the distal end hard portion 21 is described later. The distal end hard portion 21 functions as an insertion end portion of the insertion portion 20.

The bending portion 23 bends to a desired direction, for example, up, down, right, and left, in response to the operation on a bending operation portion 37. The position and the direction of the distal end hard portion 21 are changed by bending the bending portion 23. Thus, an observation target is irradiated with illumination light (not illustrated) and captured in an observation visual field. The observation target is, for example, a diseased part or a lesion site in a subject (a body cavity, etc.).

The flexible tube portion 25 has desirable flexibility. As a result, the flexible tube portion 25 is bent by external force. The flexible tube portion 25 is a tubular member extending from a body portion 31 (described later) in the operation portion 30.

[Operation Portion 30]

As illustrated in FIG. 1A, the operation portion 30 includes the body portion 31 from which the flexible tube portion 25 extends, a grasping portion 33 which is coupled to a proximal end portion of the body portion 31 and configured to be held by an operator of the endoscope 10, and a universal cord 41 which is connected to the grasping portion 33.

[Grasping Portion 33]

As illustrated in FIG. 1A, the grasping portion 33 includes a treatment instrument insertion portion 35, the bending operation portion 37 which is operated to bend the bending portion 23, and switch portions 39. The treatment instrument insertion portion 35 is disposed on a distal end portion of the grasping portion 33. The bending operation portion 37 and the switch portions 39 are disposed on a proximal end portion of the grasping portion 33.

[Treatment Instrument Insertion Portion 35]

As illustrated in FIG. 1A, the treatment instrument insertion portion 35 is branched from the grasping portion 33. As a result, the center axial direction of the treatment instrument insertion portion 35 is slanted with respect to the center axial direction of the grasping portion 33.

As illustrated in FIG. 1A, the treatment instrument insertion portion 35 has a treatment instrument insertion hole portion 35a which is disposed at an end portion of the treatment instrument insertion portion 35 and which is used to insert a medical instrument (not illustrated) into the endoscope 10.

The treatment instrument insertion hole portion 35a communicates with a proximal end portion of a treatment instrument insertion channel (not illustrated). The treatment instrument insertion channel is disposed in the insertion portion 20 from the flexible tube portion 25 to the distal end hard portion 21 through the bending portion 23. A distal end portion of the treatment instrument insertion channel communicates with a treatment instrument opening portion 130a (refer to FIG. 1B) disposed in the distal end hard portion 21. The medical instrument insertion hole portion 35a is an insertion hole through which the treatment instrument is inserted into the treatment instrument insertion channel.

[Bending Operation Portion 37]

As illustrated in FIG. 1A, the bending operation portion 37 includes a right/left bending operation knob 37a which bends the bending portion 23 in the right and the left directions, an up/down bending operation knob 37b which bends the bending portion 23 in the up and the down directions, and a bend-fixing knob such as a fixing knob 37c which fixes the position of the bending portion 23 after having been bent.

[Switch Portions 39]

As illustrated in FIG. 1A, the switch portions 39 include a suction switch 39a, an air/water supplying switch 39b, and various switch portions 39c for endoscope image capturing. The suction switch 39a, the air/water supplying switch 39b, and the various switch portions 39c are operated by the operator with a hand when the operator grips the grasping portion 33.

The suction switch 39a is operated to suction mucus, fluid, and the like with the endoscope 10 from the treatment instrument opening portion 130a serving as a suction opening portion through the treatment instrument insertion channel serving as a suction channel.

The air/water supplying switch 39b is operated when fluid is fed with air from an air supplying tube (not illustrated) and an air/water supplying tube (not illustrated), and fluid is fed with water from a water supplying tube (not illustrated) and the air/water supplying tube, in order to ensure an observation visual field 101 (refer to FIG. 1D) of an observation unit at the distal end hard portion 21. The fluid includes water and gas.

The air supplying tube, the water supplying tube, and the air/water supplying tube are disposed in the endoscope 10 from the insertion portion 20 to the universal cord 41 through the body portion 31 and the grasping portion 33.

[Universal Cord 41]

As illustrated in FIG. 1A, the universal cord 41 extends from a side surface of the grasping portion 33. The universal cord 41 includes a connector 41a which can be attached to and removed from the control apparatus 14.

[Distal End Cap for Endoscope (Hereinafter Cap 50)]

As illustrated in FIG. 1A, FIG. 1B, and FIG. 1C, the endoscope 10 further includes a cap 50 which is removably attached to the distal end hard portion 21 serving as the distal end portion of the insertion portion 20. The cap 50 has a tubular shape, for example, a substantially cylindrical shape. The cap 50 is formed of a soft material such as resin.

As illustrated in FIG. 1A and FIG. 1B, the cap 50 includes a proximal end portion 51 which is removably attached to the distal end portion of the distal end hard portion 21, and a distal end portion 53 which is disposed at a position so that the distal end portion 53 protrudes forward from a distal end surface 21a on the distal end hard portion 21 when the proximal end portion 51 is attached to the distal end portion of the distal end hard portion 21. That is, the cap 50 is removably attached to the distal end hard portion 21 so that the proximal end portion 51 as an end portion of the cap 50 is attached to the distal end hard portion 21 and the distal end portion 53 as another end of the cap 50 is positioned to protrude forward from the distal end surface 21a on the distal end hard portion 21 in the axial direction of the cap 50. In addition, the proximal end portion 51 functions as an attachment portion to attach the cap 50 to the distal end hard portion 21.

As illustrated in FIG. 1B, the distal end surface 21a includes, for example, an observation window 100a of the observation unit which observe the subject, and an illumination window 110a of an illumination unit which illuminate the subject with illumination light. The distal end surface 21a further includes an air/water supplying nozzle 120a which supply air and water toward the observation window 100a and the treatment instrument opening portion 130a from which the treatment instrument protrudes.

[Concave Portion 53a of Cap 50]

The protruding amount of the distal end portion 53 which protrude from the distal end surface 21a, in other words, the protruding height of the cap 50 is, for example, set to an optimum distance for close observation. Specifically, the distal end hard portion 21 includes the observation unit and the illumination unit. As illustrated in FIG. 1D, the observation visual field 101 of the observation unit has a non-circular shape such as a substantially octagonal shape. The following describes a case where a cylindrical cap 50 illustrated in FIG. 1E that includes a distal end portion 53, on which a concave portion 53a and a convex portion 53b are not disposed, is attached to the distal end hard portion 21. The cap 50 illustrated in FIG. 1E differs from the cap 50 of the present embodiment. In this case, as illustrated in FIG. 1F, certain parts 50a on the distal end of the cap 50 can enter both sides of the observation visual field 101 and interfere with the observation visual field 101. Although no drawing is provided, the certain parts 50a on the distal end of the cap 50 can enter both sides of an illumination range and interfere with the illumination range.

For these reasons, as illustrated in FIG. 1B and FIG. 1D, the cap 50 is formed so that the distal end portion 53 protruding from the distal end surface 21a does not block the observation visual field 101 of the observation unit and the illumination range of the illumination unit when the cap 50 is attached to the distal end hard portion 21. Specifically, as illustrated in FIG. 1B, FIG. 1C, and FIG. 1D, the certain parts 50a on the distal end of the cap 50 are recessed so that the cap 50 does not enter the observation visual field 101 and the illumination range. Therefore, as illustrated in FIG. 1B, FIG. 1C, and FIG. 2A, for example, the cap 50 includes concave portions 53a which is disposed on the distal end portion 53. The concave portions 53a are disposed (recessed) from the distal end portion 53 toward the proximal end portion 51 in the axial direction of the cap 50. The concave portions 53a may be adjacent to the observation unit in the radial direction of the cap 50 so that the concave portions 53a overlap the observation visual field 101 of the observation unit which is disposed on the distal end hard portion 21. The concave portions 53a may be adjacent to the illumination unit in the radial direction of the cap 50 so that the concave portions 53a overlap the illumination range of the illumination unit which is disposed on the distal end hard portion 21. It is sufficient if at least one of the above-mentioned overlaps is implemented. For example, the concave portions 53a are disposed so as to be adjacent to the observation window 100a and the illumination window 110a. Note that it is sufficient if at least a part of the concave portions 53a is disposed so as to be adjacent to the observation window 100a and the illumination window 110a in accordance with the shape of a mask in an endoscopic observation image, areas in which illumination light is allowed to be blocked, areas in which illumination light should not be blocked, or the divergence of the observation visual field 101 and the illumination light. The concave portions 53a are disposed on the certain parts 50a of the cap 50 in the axis circumferential direction of the cap 50. Multiple concave portions 53a are disposed in the axis circumferential direction of the cap 50.

The concave portions 53a may be formed so as not to enter either the observation visual field or the illumination range.

[Convex Portions 53b of Cap 50]

As illustrated in FIG. 1B, FIG. 1C, and FIG. 2A, the convex portions 53b are disposed so as to be adjacent to the concave portions 53a in the axis circumferential direction of the cap 50. That is, the concave portions 53a and the convex portions 53b are alternately disposed in the axis circumferential direction of the cap 50. The concave portions 53a and the convex portions 53b are disposed on an edge portion on the distal end of the cap 50. The concave portions 53a and the convex portions 53b function as the distal end of the cap 50.

The shape, the height, and other attributes of the concave portions 53a and the convex portions 53b are desirably set in accordance with the shape of the mask in the endoscopic observation image, areas in which illumination light is allowed to be blocked, areas in which illumination light should not be blocked, or the divergence of the observation visual field 101 and the illumination light.

[Distal End Surface 53c of Cap 50]

As illustrated in FIG. 1B and FIG. 1C, in the distal end portion 53 of the cap 50 which having the concave portions 53a and the convex portions 53b mentioned above, a distal end surface 53c of the distal end portion 53 is formed in, for example, a wave shape in the axis circumferential direction of the cap 50. Consequently, the protruding amount of the distal end of the cap 50 varies partially. A ridge portion 53d of the distal end surface 53c is smooth in the axis circumferential direction of the cap 50. That is, in the axis circumferential direction of the cap 50, the concave portions 53a smoothly continue to the convex portions 53b without steps.

When the concave portions 53a and the convex portions 53b are disposed, for example, the strength of the convex portions 53b that are parts of the cap 50 is lower than that of the proximal end portion 51 that is another part of the cap 50. In other words, the strength of the cap 50 is uneven. In the present embodiment, therefore, the cap 50 includes a low-strength portion 55 and a high-strength portion 57 having strength higher than that of the low-strength portion 55 to reinforce the strength of at least the convex portions 53b.

[Low-Strength Portion 55]

As illustrated in FIG. 1B, FIG. 1C, and FIG. 2A, the low-strength portion 55 is disposed on a same line as, for example, at least the concave portions 53a in the axial direction of the cap 50. The low-strength portion 55 is continuously disposed between the distal end portion 53 and the proximal end portion 51 in the axial direction of the cap 50. For example, the low-strength portion 55 is disposed linearly. As a result, the low-strength portion 55 is disposed at the same position to the observation unit in the axis circumferential direction of the cap 50 when the cap 50 is attached to the distal end hard portion 21. In other words, the low-strength portion 55 is disposed so as to be adjacent to the observation unit in the radial direction of the cap 50 when the cap 50 is attached to the distal end hard portion 21. The same applies to the low-strength portion 55 and the illumination unit.

In the present embodiment, as illustrated in FIG. 2A, for example, the low-strength portion 55 has a substantially C-shaped section. For example, a width of the low-strength portion 55 is even. The low-strength portion 55 includes the concave portions 53a. As a result, the distal end portion 53 to which the low-strength portion 55 is disposed includes the bottoms of the concave portions 53a. The low-strength portion 55 includes the convex portions 53b. As a result, the distal end portion 53 to which the low-strength portion 55 is disposed includes the distal ends of the convex portions 53b.

[High-Strength Portion 57]

As illustrated in FIG. 1B, FIG. 1C, and FIG. 2A, the high-strength portion 57 is disposed on at least a portion excluding the concave portions 53a in the axis circumferential direction of the cap 50. In the present embodiment, the high-strength portion 57 is disposed only in a portion excluding the concave portions 53a, for example, only in a portion including one of the convex portions 53b in the axis circumferential direction of the cap 50. The high-strength portion 57 is continuously disposed between the distal end portion 53 and the proximal end portion 51 in the axial direction of the cap 50. For example, the high-strength portion 57 is disposed linearly. The high-strength portion 57 is disposed to include the convex portion 53b, and therefore, the distal end portion 53 to which the high-strength portion 57 is disposed includes the distal end of the convex portion 53b. The high-strength portion 57 is disposed on the same line as the convex portion 53b in the axial direction of the cap 50. For example, the high-strength portion 57 is disposed so as to be adjacent to the low-strength portion 55 in the axis circumferential direction of the cap 50. Accordingly, For example, the high-strength portion 57 is disposed so as to be adjacent to the observation unit and the observation window 100a in the radial direction of the cap 50 when the cap 50 is attached to the distal end hard portion 21. For example, the high-strength portion 57 is disposed so as to be adjacent to the illumination unit and the illumination window 110a in the axis circumferential direction of the cap 50 when the cap 50 is attached to the distal end hard portion 21. For example, a width of the high-strength portion 57 in the axis circumferential direction of the cap 50 is even from the distal end portion 53 to the proximal end portion 51 in the axial direction of the cap 50.

For example, the one high-strength portion 57 is disposed to correspond to one of the convex portions 53b. As illustrated in FIG. 1B, FIG. 1C, FIG. 2A, FIG. 2B, and FIG. 2C, it is sufficient if at least one high-strength portion 57 is disposed. A plurality of high-strength portions 57 may be disposed in the axis circumferential direction of the cap 50.

As illustrated in FIG. 2A, for example, the low-strength portion 55 includes a thin wall portion 55a and the high-strength portion 57 includes a thick wall portion 57a. In this case, as illustrated in FIG. 2A and FIG. 2C, for example, the thick wall portion 57a is formed by making an outer diameter of the cap 50 at the thick wall portion 57a larger than that of the cap 50 at the thin wall portion 55a. An inner diameter of the cap 50 at the thick wall portion 57a is equal to that of the cap 50 at the thin wall portion 55a. Alternatively, as illustrated in FIG. 2B, the thick wall portion 57a is formed by making the inner diameter of the cap 50 at the thick wall portion 57a smaller than that of the cap 50 at the thin wall portion 55a. The outer diameter of the cap 50 at the thick wall portion 57a is equal to that of the cap 50 at the thin wall portion 55a.

[Slip Prevention Portions 51a]

As illustrated in FIG. 3A and FIG. 3B, the proximal end portion 51 includes slip prevention portions 51a disposed on an inner circumferential surface of the proximal end portion 51 to prevent the proximal end portion 51 from slipping off of the distal end hard portion 21 when the cap 50 is attached to the distal end hard portion 21. The slip prevention portions 51a prevent the proximal end portion 51 attached to the distal end hard portion 21 from moving in the axial direction and the axis circumferential direction of the cap 50 when the cap 50 is attached to the distal end hard portion 21. For example, the slip prevention portions 51a serve as protruding portions protruding toward the inside of the proximal end portion 51 from the inner circumferential surface of the proximal end portion 51. The slip prevention portions 51a are disposed in order to increase a friction area between the inner circumferential surface of the proximal end portion 51 and an outer circumferential surface of the distal end hard portion 21. For example, the slip prevention portions 51a each have a quadrangular prism shape illustrated in FIG. 3A or a triangular prism shape illustrated in FIG. 3B. The slip prevention portions 51a are disposed apart from each other with an even distance in the axis circumferential direction of the cap 50. Note that, for example, the slip prevention portions 51a may be disposed on the entire circumference of the proximal end portion 51 in the axis circumferential direction of the cap 50. In this case the slip prevention portions 51a have, for example, a ring shape.

[Marker 51b]

As illustrated in FIG. 1B, FIG. 3A, FIG. 3B, and FIG. 3C, the proximal end portion 51 includes a marker 51b which is disposed in order to align the distal end hard portion 21 and the cap 50 in the axis circumferential direction of the distal end hard portion 21 and the axis circumferential direction of the cap 50 so that the concave portions 53a are disposed so as to be adjacent to the observation unit and the illumination unit as described above. When the marker 51b is disposed at a desired portion in the axis circumferential direction of the distal end hard portion 21, the concave portions 53a are disposed so as to be adjacent to the observation unit and the illumination unit as described above. For example, the marker 51b is formed by disposing a cut-out in a part of the proximal end portion 51.

[Penetration Prevention Portions 59]

As illustrated in FIG. 3C, the cap 50 includes penetration prevention portions 59 which are disposed on an inner circumferential surface of the cap 50 to prevent the distal end hard portion 21 from penetrating through the cap 50 in the axial direction of the cap 50 in a manner that the distal end surface 21a of the distal end hard portion 21 abuts on the penetration prevention portions 59. The penetration prevention portions 59 function as protruding portions protruding from the inner circumferential surface of the cap 50 toward the inside of the cap 50. For example, the protruding members each include a rib.

Note that, as illustrated in FIG. 3C, the penetration prevention portions 59 are disposed on certain parts on the cap 50 in the axis circumferential direction of the cap 50. Specifically, the penetration prevention portions 59 are disposed so as to not block the observation visual field 101 of the observation unit and the illumination range of the illumination unit. For this reason the penetration prevention portions 59, for example, are disposed on the inner circumferential surfaces of the convex portions 53b. The penetration prevention portions 59 may be disposed at the same positions as those of the bottoms of the concave portions 53a in the axial direction of the cap 50. That is, the penetration prevention portions 59 and the bottoms of the concave portions 53a may be disposed on the same plane in the radial direction of the cap 50. The cap 50 may be molded in a manner that the penetration prevention portions 59 and the bottoms of the concave portions 53a are integrated. For example, the penetration prevention portions 59 may be disposed on the entire circumference of the distal end portion 53 in the axis circumferential direction of the cap 50.

[Operation]

The cap 50 is attached to the distal end hard portion 21 so that the distal end hard portion 21 and the cap 50 are aligned with each other by the marker 51b in the axis circumferential direction of the distal end hard portion 21 and the axis circumferential direction of the cap 50.

At this point, the slip prevention portions 51a prevent the proximal end portion 51 from slipping off from the distal end hard portion 21 and prevent the proximal end portion 51 attached to the distal end hard portion 21 from moving in the axial direction and the axis circumferential direction of the cap 50.

The penetration prevention portions 59 prevent the distal end hard portion 21 from penetrating through the cap 50 in a manner that the distal end surface 21a of the distal end hard portion 21 abuts on the penetration prevention portions 59.

With this configuration, the cap 50 including the concave portions 53a, the low-strength portion 55, and the high-strength portion 57 is positioned relative to the distal end hard portion 21 in the axis circumferential direction of the cap 50 and the axial direction of the cap 50.

Note that, in a case different from the present embodiment where the high-strength portion 57 is not disposed, only the low-strength portion 55 is disposed, a desired strength for the cap 50 may not be able to be ensured. As a result, the cap 50 may deform and cannot be attached to the distal end hard portion 21 easily when the cap 50 is attached to the distal end hard portion 21.

However, in the present embodiment, the high-strength portion 57 is disposed, so that the desired strength of the cap 50 is ensured. As a result, the cap 50 is prevented from deforming and is easily attached to the distal end hard portion 21 when the cap 50 is attached to the distal end hard portion 21.

The concave portions 53a are disposed. This configuration prevents certain parts of 50a on the distal end of the cap 50 from blocking the observation visual field 101 and the illumination range.

When the cap 50 is pressed against the wall of a lumen, the convex portions 53b abut on the wall first. If the strength of the convex portions 53b is low, the convex portions 53b may collapse toward the inside of the cap 50. The collapsed convex portions 53b may block the observation visual field 101 and can block the illumination light.

By contrast, in the present embodiment, the high-strength portion 57 can ensure the desired strength of the convex portions 53b because the high-strength portion 57 is disposed so as to include the convex portions 53b. Thus the convex portions 53b are prevented from collapsing toward the inside of the cap 50. In this manner the high-strength portion 57 ensures the strength required for the convex portions 53b in the cap 50. Accordingly, the convex portions 53b are prevented from blocking the observation visual field 101 and the illumination light.

The high-strength portion 57 eliminates the need for a part of the cap 50 to protrude to the outside of the cap 50, and for an increase in the overall wall thickness of the cap 50 in the radial direction of the cap. Therefore, the overall outer diameter of the cap 50 is prevented from increasing, so that the insertion-removal operability of the insertion portion 20 is prevented from decreasing.

The high-strength portion 57 is continuously disposed linearly between the distal end portion 53 and the proximal end portion 51 in the axial direction of the cap 50. As a result, the cap 50 ensures the desired strength in the axial direction of the cap 50.

The high-strength portion 57 may include a plurality of high-strength portions 57 disposed in the axis circumferential direction of the cap 50. As a result, the cap 50 ensures the desired strength in the axis circumferential direction of the cap 50.

The high-strength portion 57 includes the thick wall portion 57a. For this reason, the cap 50 is molded easily. The high-strength portion 57 includes the thick wall portion 57a and is disposed as described above. Accordingly, the overall wall thickness of the cap 50 is prevented from increasing, preventing the overall outer diameter of the cap 50 from increasing.

[Effects]

With the above-described manner, in the present embodiment, the high-strength portion 57 can ensure the desired strength of the convex portion 53b having low strength. Consequently, in the present embodiment, the convex portion 53b can be prevented from collapsing toward the inside of the cap 50. Thus, in the present embodiment, the high-strength portion 57 can ensure the strength required for the convex portion 53b in the cap 50. Consequently, in the present embodiment, the convex portion 53b can be prevented from blocking the observation visual field 101 and the illumination light.

In the present embodiment, the high-strength portion 57 can eliminate the need for a part of the cap 50 to protrude to the outside of the cap 50, and for an increase in the overall wall thickness of the cap 50 in the radial direction of the cap 50. Consequently, in the present embodiment, the overall outer diameter of the cap 50 can be prevented from increasing, thereby preventing the decrease of the insertion-removal operability of the insertion portion 20.

In the present embodiment, the high-strength portion 57 is continuously disposed linearly between the distal end portion 53 and the proximal end portion 51 in the axial direction of the cap 50. Consequently, in the present embodiment, the cap 50 can ensure the desired strength in the axial direction of the cap 50.

In the present embodiment, high-strength portion 57 may include a plurality of high-strength portions 57 disposed in the axis circumferential direction of the cap 50. Consequently, in the present embodiment, the cap 50 can ensure the desired strength in the axis circumferential direction of the cap 50.

In the present embodiment, the high-strength portion 57 includes the thick wall portion 57a. For this reason, in the present embodiment, the cap 50 can be molded easily. In the present embodiment, the high-strength portion 57 includes the thick wall portion 57a and is disposed as described above. Consequently, in the present embodiment, the overall wall thickness of the cap 50 can be prevented from increasing, thereby preventing the overall outer diameter of the cap 50 from increasing.

In the present embodiment, the high-strength portion 57 is disposed, so that the desired strength of the cap 50 is ensured. Consequently, in the present embodiment, the cap 50 can be prevented from deforming and can be easily attached to the distal end hard portion 21 when the cap 50 is attached to the distal end hard portion 21.

In the present embodiment, the concave portions 53a are disposed. Consequently, in the present embodiment, certain parts 50a of the distal end can be prevented from blocking the observation visual field 101 and the illumination range.

In the present embodiment, with the marker 51b, the cap 50 and the distal end hard portion 21 can be easily aligned with each other in the axis circumferential direction of the cap 50 and the axis circumferential direction of the distal end hard portion 21.

In the present embodiment, the slip prevention portions 51a can prevent the cap 50 from slipping off from the distal end hard portion 21, thereby preventing the cap 50 attached to the distal end hard portion 21 from moving in the axial direction and the axis circumferential direction of the cap 50.

In the present embodiment, the penetration prevention portions 59 can prevent the distal end hard portion 21 from penetrating through the cap 50 in a manner that the distal end surface 21a of the distal end hard portion 21 abuts on the penetration prevention portions 59.

With the above-described configuration, in the present embodiment, the cap 50 including the concave portions 53a, the low-strength portion 55, and the high-strength portion 57 can be positioned relative to the distal end hard portion 21 in the axis circumferential direction and the axial direction of the cap 50.

For example, in the present embodiment, the width of the high-strength portion 57 is even. Consequently, in the present embodiment, the cap 50 can ensure the desired strength in the axis circumferential direction of the cap 50.

Note that the high-strength portion 57 may be disposed only on the convex portions 53b. The high-strength portion 57 may simply be continuously disposed linearly between the distal end portion 53 and the proximal end portion 51 in the axial direction of the cap 50 so as to include the concave portions 53a.

In the present embodiment, the above-mentioned configuration can provide an endoscope apparatus 5 including the cap 50 attached to the insertion end portion (the distal end hard portion 21) disposed on the endoscope 10. In other words, in the present embodiment, the above-mentioned configuration can provide the endoscope apparatus 5 including an endoscope insertion portion to which the cap 50 is attached.

The following briefly describes modifications of the present embodiment.

[First Modification]

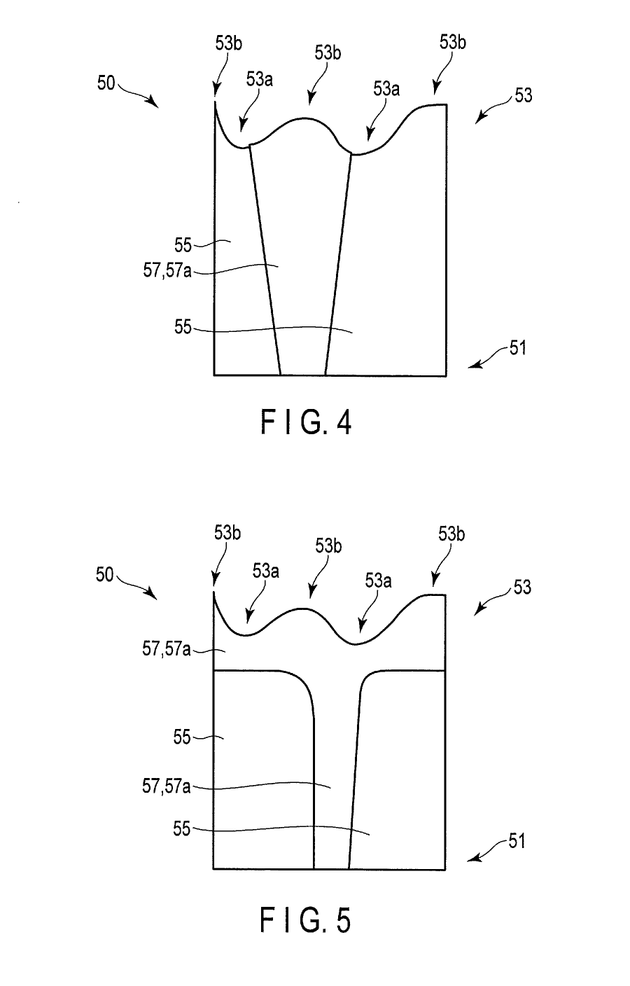

As illustrated in FIG. 4, the width of the high-strength portion 57 may be gradually decreased from the distal end portion 53 to the proximal end portion 51 in the axial direction of the cap 50. The high-strength portion 57 includes the thick wall portion 57a similar to the first embodiment.

Consequently, in the present modification, the desired strength required for a part can be ensured, and the thick wall portion 57a can be reduced.

Note that, in a case where the high-strength portion 57 includes the thick wall portion 57a, the thickness of the thick wall portion 57a may gradually be decreased from the distal end portion 53 to the proximal end portion 51.

[Second Modification]

As illustrated in FIG. 5, the high-strength portion 57 may be disposed on the distal end portion 53 and disposed on the entire circumference of the distal end portion 53 in the axis circumferential direction of the cap 50 so as to include the concave portions 53a.

Consequently, in the present modification, the desired strength required for a part can be ensured, and the thick wall portion 57a can be reduced.

[Third Modification]

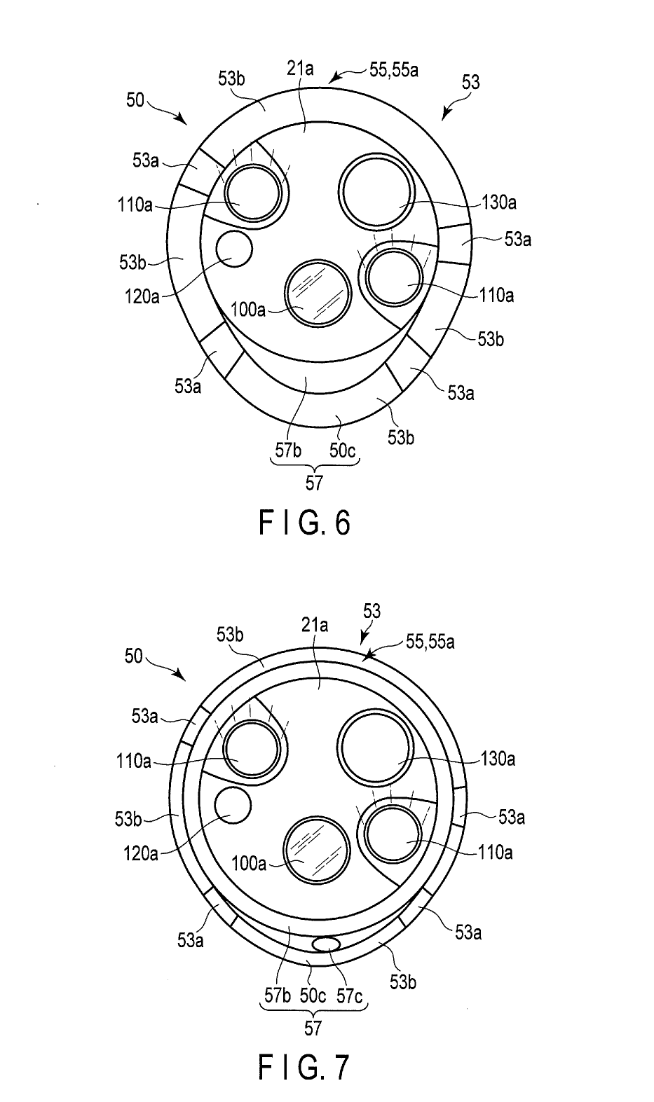

As illustrated in FIG. 6, the low-strength portion 55 functions as the cap 50.

As illustrated in FIG. 6, the high-strength portion 57 includes a part 50c of the cap 50 and a reinforcing member 57b which is disposed on, for example, an inner circumferential surface of the part 50c, is different from the cap 50 and reinforces the strength of the part 50c. The strength of the reinforcing member 57b may be equal to or different from that of the cap 50. The reinforcing member 57b has a pillar shape with a substantially arc-shaped cross-section. The reinforcing member 57b is fixed to a part on the inner circumferential surface of the cap 50. The reinforcing member 57b may be formed of a material that is the same as or different from that of the cap 50.

With this configuration, in the present modification, the desired strength required for a part can be ensured, and the amount of work on the cap 50 itself can be reduced.

Note that it is sufficient if the reinforcing member 57b is disposed on at least one of the inner circumferential surface and an outer circumferential surface of the cap 50. The reinforcing member 57b is disposed so as to abut on at least one of the inner circumferential surface and an outer circumferential surface of the cap 50.

Although no drawing is provided, the high-strength portion 57 may include the part 50c of the cap 50 and a coating coated on the part 50c of the cap 50 so as to increase the strength of the part 50c of the cap 50. For example, the coating may contain a silicone adhesive.

[Fourth Modification]

As illustrated in FIG. 7, the reinforcing member 57b may have a tubular shape, for example, a cylindrical shape and may be disposed in the cap 50. The reinforcing member 57b is disposed between the cap 50 and the distal end hard portion 21 in the radial direction of the distal end hard portion 21 so that the distal end hard portion 21 is inserted in the reinforcing member 57b and an outer circumferential surface of the distal end hard portion 21 closely contacts an inner circumferential surface of the reinforcing member 57b. Therefore, the high-strength portion 57 may include a sandwiched member 57c that is disposed between the cap 50 and the reinforcing member 57b and sandwiched between the cap 50 and the reinforcing member 57b in the radial direction of the cap 50. For example, the sandwiched member 57c has a rod shape.

With this configuration, in the present modification, the desired strength required for a part can be ensured, and the amount of work on the cap 50 itself can be reduced.

[Fifth Modification]

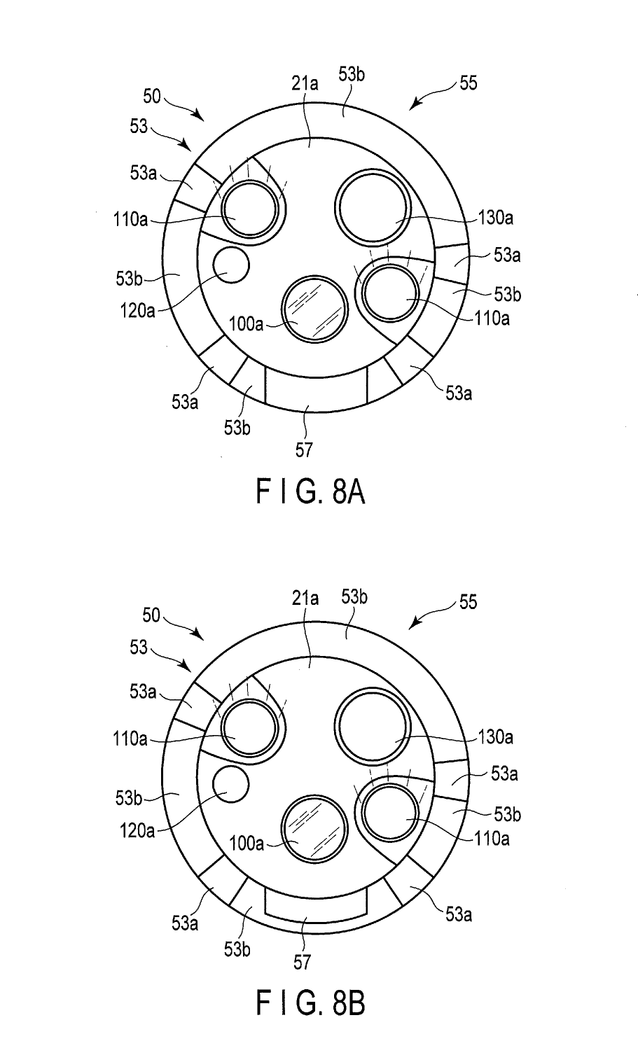

As illustrated in FIG. 8A, the low-strength portion 55 and the high-strength portion 57 are formed of materials different from each other. In this case, the low-strength portion 55 is formed of a soft material, but the high-strength portion 57 is formed of a hard material. For example, such materials are resin. In addition, the low-strength portion 55 and the high-strength portion 57 are integrally molded to one another and function as the cap 50.

Note that, as illustrated in FIG. 8B, the high-strength portion 57 may be integrally molded with the low-strength portion 55 by insert molding, and the low-strength portion 55 and the high-strength portion 57 may function as the cap 50.

With this configuration, in the present modification, the wall thickness of the cap 50 can be even, thereby preventing the decrease of the insertion-extraction operability of the insertion portion 20.

[Sixth Modification]

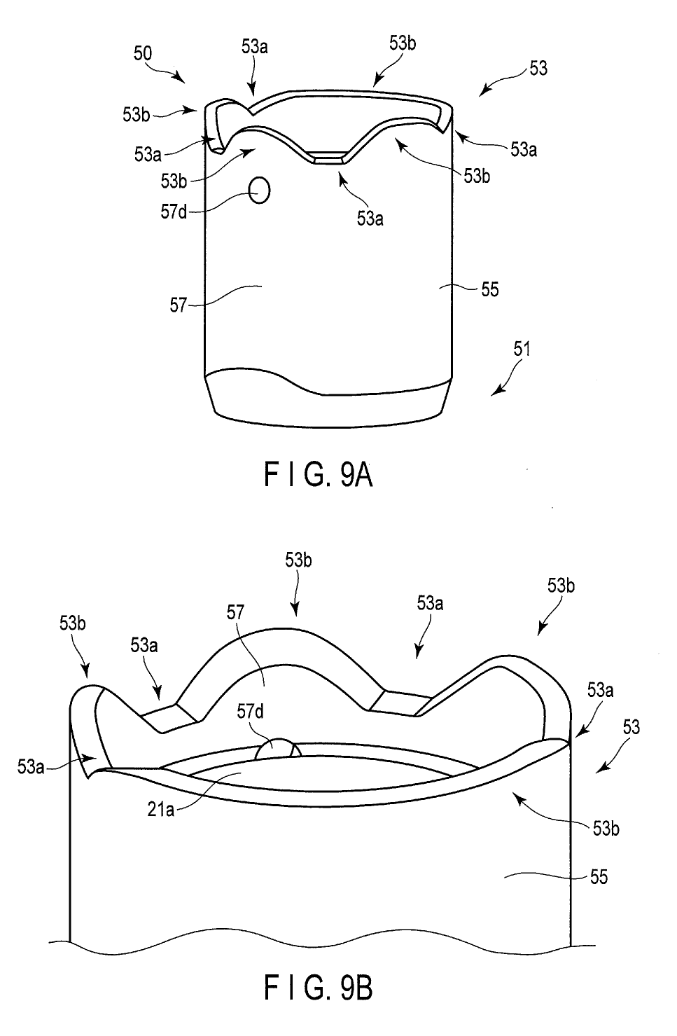

As illustrated in FIG. 9A and FIG. 9B, the high-strength portion 57 may include a through hole portion 57d penetrating through the cap 50 in the radial direction of the cap 50. For example, the through hole portion 57d allows fluid for washing the observation window of the observation unit to flow out from the inside of the cap 50 to the outside. The through hole portion 57d is disposed to the distal end portion 53 so that at least a part of the through hole portion 57d is not covered with a side surface of the distal end hard portion 21, and at least a part of the through hole portion 57d is exposed from the distal end surface 21a when the cap 50 is attached to the distal end hard portion 21. For example, the through hole portion 57d is disposed to a proximal end portion of the convex portions 53b.

With this configuration, in the present modification, the through hole portion 57d allows fluid accumulated in the cap 50 to flow out to the outside when the cap 50 is attached to the distal end hard portion 21. In the present modification, if the through hole portion 57d is disposed to the low-strength portion 55, the strength of the low-strength portion 55 is further reduced. However, the through hole portion 57d is disposed to the high-strength portion 57, so that the desired strength required for a part can be ensured.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A distal end cap for an endoscope which is removably attached in an axial direction to an insertion end portion of an insertion portion disposed in the endoscope, the distal end cap comprising:
a cap proximal end portion which is disposed at a proximal end side of the distal end cap in the axial direction and attached to the insertion end portion;
a cap distal end portion which is disposed at a distal end side of the distal end cap in the axial direction, and is disposed at a position so that the cap distal end portion protrudes from an distal end surface of the insertion end portion when the cap proximal end portion is attached to the insertion end portion;
a concave portion which is disposed on the cap distal end portion from the distal end side toward the proximal end side in the axial direction;
a low-strength portion which is disposed on a same line as the concave portion in the axial direction and continuously disposed between the cap distal end portion and the cap proximal end portion in the axial direction; and
at least one high-strength portion having strength higher than that of the low-strength portion, the high-strength portion being disposed on at least a portion excluding the concave portion in an axis circumferential direction of an axis, and continuously disposed from the cap distal end portion to the cap proximal end portion in the axial direction.

2. The distal end cap for an endoscope according to claim 1, wherein a plurality of high-strength portions disposed in the axis circumferential direction.

3. The distal end cap for an endoscope according to claim 1, wherein a width of the high-strength portion in the axis circumferential direction is even from the cap distal end portion to the cap proximal end portion in the axial direction, or gradually decreases from the cap distal end portion to the cap proximal end portion in the axial direction.

4. The distal end cap for an endoscope according to claim 1, wherein the high-strength portion is disposed on the entire circumference of the cap distal end portion in the axis circumferential direction so as to include the concave portion.

5. The distal end cap for an endoscope according to claim 1, wherein
the low-strength portion includes a thin wall portion, and
the high-strength portion includes a thick wall portion.

6. The distal end cap for an endoscope according to claim 1, wherein the high-strength portion includes a reinforcing member which is different from the distal end cap to reinforce strength of at least one of an inner circumferential surface and an outer circumferential surface of the distal end cap for an endoscope.

7. The distal end cap for an endoscope according to claim 6, wherein the reinforcing member has a pillar shape with a substantially arc cross-shaped cross-section and is disposed so as to abut on the inner circumferential surface or the outer circumferential surface.

8. The distal end cap for an endoscope according to claim 6, wherein
the reinforcing member disposed on the inner circumferential surface and having a tubular shape,
the high-strength portion includes a sandwiched member which is sandwiched between the inner circumferential surface and the reinforcing member.

9. The distal end cap for an endoscope according to claim 1, wherein
the high-strength portion is formed of a material different from the low-strength portion and harder than that of the low-strength portion, and
the low-strength portion and the high-strength portion are integrally molded to one another, or molded by insert molding.

10. The distal end cap for an endoscope according to claim 1, wherein the high-strength portion is disposed so as to be adjacent to an observation unit disposed on the insertion end portion in a radial direction of the distal end cap for an endoscope when the distal end cap for an endoscope is attached to the insertion end portion.

11. The distal end cap for an endoscope according to claim 1, wherein the concave portion is disposed so as to be adjacent to an observation unit disposed on the insertion end portion in a radial direction of the distal end cap for an endoscope so that the concave portion overlaps an observation visual field of the observation unit when the distal end cap for an endoscope is attached to the insertion end portion.

12. The distal end cap for an endoscope according to claim 1, wherein the concave portion is disposed so as to be adjacent to an illumination unit disposed on the insertion end portion in a radial direction of the distal end cap for an endoscope so that the concave portion overlaps an illumination range of the illumination unit when the distal end cap for an endoscope is attached to the insertion end portion.

13. An endoscope apparatus comprising:
an endoscope; and
the distal end cap for an endoscope according to claim 1 removably attached to an insertion end portion disposed on the endoscope.
